(19) United States
(12) Patent Application Publication
(10) Pub. No.: US 2025/0302900 A1
(43) Pub. Date: Oct. 2, 2025

(54) Method of Treating Heart Failure and Traditional Chinese Medicine Composition for Treating Heart Failure

(71) Applicant: Guangzhou Xitao Biosciences Co., Ltd., Guangzhou, Guangdong (CN)

(72) Inventors: Haoyu Zeng, Guangzhou, Guangdong (CN); Sihua Hong, Guangzhou, Guangdong (CN); Jiexing Chen, Guangzhou, Guangdong (CN)

(21) Appl. No.: 18/700,745

(22) PCT Filed: Mar. 27, 2024
(86) PCT No.: PCT/CN2024/083958

§ 371 Date: Apr. 12, 2024

(51) Int. Cl. A61K 36/18; A61K 35/413; A61P 9/04

(52) U.S. Cl. CPC A61K 36/18; A61K 35/413; A61P 9/04

(57) ABSTRACT

Provided in the present disclosure is a method of treating heart failure and a traditional Chinese medicine composition for treating heart failure, the method of treating heart failure including administrating to a subject an amount of a traditional Chinese medicine composition, in which the traditional Chinese medicine composition includes following components in terms of parts by weight: 46-53.6 parts of Semen Myristicae, 42.4-49.2 parts of Radix Vladimiriae, 98.4-111.6 parts of Fructus Chebulae, 50.8-65.2 parts of Stigma Croci, 17.2-26 parts of Bovis Calculus, and 9.2-13.6 parts of bear bile powder. The aforementioned traditional Chinese medicine composition enhances the efficacy of the medicine and broadens the scope of treatment by ingeniously combining a variety of traditional Chinese medicinal ingredients, which achieves concurrent regulation of qi, blood, yin and yang, and simultaneous treatment of the organs, effectively exerting therapeutic effects on heart failure.

### FIELD

The present disclosure relates to the technical field of traditional Chinese medicines and, particularly, to an application of a traditional Chinese medicine composition in a preparation of a drug for treating heart failure.

### BACKGROUND

Heart failure is an end stage of various cardiovascular diseases, with a high prevalence rate. Patients typically require lifelong medication once diagnosed, which significantly impacts their quality of life. Current clinical treatments for heart failure mainly include angiotensin-converting enzyme inhibitors, beta-blockers, aldosterone receptor antagonists, and diuretics. However, long-term use of these drugs may lead to adverse effects such as hypotension, fluid depletion, electrolyte imbalances, and may even lead to increased mortality rates in patients.

Traditional Chinese medicine attributes heart failure to the categories of “edema” and “dyspnea”, and terms it as “heart failure”. From the perspective of traditional Chinese medicine, although the pathological site of heart failure lies in the heart, manifesting as myocardial contraction weakness, the main pathogenesis is attributed to yang deficiency of the heart and kidneys, qi deficiency and weakness, leading to blood stagnation due to decreased blood circulation function in the heart. The predisposing group of heart failure is mostly the elderly, as the decline of body functions of the predisposing group tends to result in a vicious cycle of chronic illness, qi deficiency, yang deficiency, impaired blood circulation, and blood stagnation.

In recent years, it has been confirmed by modern pharmacology that a type of traditional Chinese medicine with the effects of benefiting qi, warming yang, circulating blood and draining water retention may increase oxygen consumption in the myocardium. The use of traditional Chinese medicine with these properties has shown superior efficacy in treating coronary heart disease and heart failure. This type of medicine consists of Chinese herbs such as Ginseng Radix et Rhizoma, Herba Leonuri, Salviae Miltiorrhizae Radix et Rhizoma, Radix Astragali, Ramulus Cinnamomi, Rhizoma Atractylodis macrocephalae, Radix Glycyrrhizae, Radix et Rhizoma Notoginseng, and Radix Angelicae Sinensis. Research on such type of medicine has shown promising effects in treating heart failure. However, with the continuous use of such type of medicine, some reports suggest the development of drug resistance, potentially leading to suboptimal treatment outcomes.

Therefore, whether it is feasible to explore a traditional Chinese medicine composition from other types of Chinese herbs that may effectively treat heart failure is a new research challenge in the art.

### SUMMARY

To address the problems and deficiencies in the prior art, provided in the present disclosure is an application of a traditional Chinese medicine composition in a preparation of a drug for treating heart failure, in which the traditional Chinese medicine composition employed is effective in treating heart failure and in improving the cardiac function of the patient by employing a variety of traditional Chinese medicinal ingredients used in combination with each other.

In accordance with a first aspect of the present disclosure, provided is an application of a traditional Chinese medicine composition in a preparation of a drug for treating heart failure, in which the traditional Chinese medicine composition includes following components in terms of parts by weight: 46-53.6 parts of Semen Myristicae, 42.4-49.2 parts of Radix Vladimiriae, 98.4-111.6 parts of Fructus Chebulae, 50.8-65.2 parts of Stigma Croci, 17.2-26 parts of Bovis Calculus, and 9.2-13.6 parts of bear bile powder.

Semen Myristicae refers to the dried seed of plant of Myristica Fragrans in the family Myristaceae, originated from “Lei's Treatise on Processing of Drugs”, pungent and warm in nature, mainly acting on the spleen, stomach and large intestine. Semen Myristicae has the efficacy of warming the interior, circulating qi, astringing the intestines, and stopping diarrhea, which may be used for symptoms such as deficiency cold of the spleen and stomach, prolonged diarrhea, distention and pain in the stomach and abdomen, reduced appetite, and vomiting. Chemical composition studies have shown that Semen Myristicae mainly contains volatile oils, phenylpropanoids, lignans, diarylalkanes, flavonoids, and other chemical components. Modern pharmacological studies have demonstrated that Semen Myristicae exhibits a variety of pharmacological activities such as anti-inflammation and analgesia, anti-oxidation, anti-bacteria, anti-tumor, and lowering glucose. Consequently, Semen Myristicae is also commonly used in traditional medicine for treating such as rheumatism, cholera, anxiety, stomach cramps, nausea, and diarrhea, as well as for aphrodisiacs and abortifacients purposes. Semen Myristicae also plays a role in treating heart disease or heart failure, although there are relatively few reports and research on this. Typically, Semen Myristicae is usually utilized in conjunction with other Chinese herbs for treating heart failure or a variety of heart diseases such as heart heyi (heyi is a disease), heart tingling, delirium, syncope, and heart panic.

Radix Vladimiriae is mainly distributed at the border of Sichuan and Tibet, and grows in alpine meadows and thickets at an altitude of more than 3,000 meters. It is a plant of the genus Dolomiaea in the family Asteraceae (Compositae). The dried roots of Radix Vladimiriae are used as medicinal parts according to “the Pharmacopoeia of the People's Republic of China”. Radix Vladimiriae is warm in nature, pungent and bitter in taste, with the efficacy of circulating qi and analgesia, clinically used for chest and hypochondrium, distension and pain in the stomach and abdomen, bowel sounds and diarrhea, and tenesmus. Various small molecule compounds have been isolated from the plants of genus Dolomiaea, with sesquiterpenes being the main chemical constituents, along with resinoids, diterpenoids, triterpenoids, and steroids. Modern pharmacological studies have shown that the pharmacological activities of this species mainly involve anti-inflammation, anti-bacteria, anti-tumor, and inhibition of gastric ulcers. There are limited reports on the use of Radix Vladimiriae in treating heart disease or heart failure, and it is also often necessary to combine it with other Chinese herbs in order to achieve certain efficacy.

Terminalia chebular Retz. is a deciduous tree belonging to the genus Terminalia in the family Combretaceae R. Br., and the dried mature fruits thereof, known as Fructus Chebulae, is commonly used in traditional Chinese medicine. The Chinese medicinal herbs Fructus Chebulae is bitter, sour, astringent in taste, neutral in nature, mainly acting on the lung and large intestine, with the efficacy of astringing the intestines and stopping diarrhea, astringing the lungs and stopping cough, used for symptoms such as prolonged diarrhea and dysentery, bleeding and prolapse of the anus, lung deficiency with dyspnea and coughing, prolonged coughing, and pain in the pharynx with hoarseness of voice. Fructus Chebulae is widely used in folk medicine in China and is even regarded as the “king of medicines” in Tibetan medicine, which may be used in treating many diseases. Fructus Chebulae contains a wide variety of chemical components, mainly containing phenolic acids, tannins, triterpenoids such as gallic acid, chebulinic acid, and arjungenin. Modern pharmacological studies have shown that extracts of Fructus Chebulae such as its aqueous extract and individual components contained therein exhibit bioactivities such as anti-bacteria, anti-oxidation, anti-tumor, anti-inflammation, toxicity-reducing and detoxifying, analgesia, cardiotonic, hepatoprotection, lowering glucose, inhibiting gastric emptying and small intestinal propulsion, promoting neovascularization, and protecting the brain and liver.

Stigma Croci commonly known as saffron, belongs to the genus Crocus L. in the family Iridaceae, cultivated in countries such as Iran, Greece, Italy, Spain, India, China, and Japan. The main medicinal part thereof is the style (female organ of flower), which has the efficacy of promoting blood circulation and transforming stasis, cooling blood and detoxifying, relieving depression and calming mind, and may be used for symptoms such as amenorrhea, postpartum blood stasis, warm toxin causing skin eruptions, melancholic with oppression of the chest, palpitations leading to confusion. Modern pharmacological studies have shown that Stigma Croci has beneficial effects on overall health. Over 100 compounds have been isolated from Stigma Croci, including terpenoids, flavonoids, and anthraquinones, and they have demonstrated therapeutic effects on such as psychiatric disorders, neurodegenerative diseases, learning and memory impairments, cardiovascular diseases, atherosclerosis, hyperlipidemia, diabetes, hypertension, gastric ulcers, fatty liver, antiepileptic and anticonvulsant.

Bovis Calculus, known as the dried gallstones of the bovine in the family Bovidae, is a precious traditional Chinese medicine. At present, there are four kinds of goods of Bovis Calculus, natural Bovis Calculus, cultivated Bovis Calculus, Bovis Calculus Artifactus and in vitro cultivated Bovis Calculus. Natural Bovis Calculus is scarce and expensive, and the production of cultivated Bovis Calculus is quite limited. Therefore, in clinical practice, in vitro cultured Bovis Calculus and Bovis Calculus Artifactus are more commonly used. First found in “the Shennong's Classic of the Materia Medica”, traditional Chinese medicine believes that natural Bovis Calculus is effective in clearing heat and transforming phlegm. Modern pharmacological studies have shown that natural Bovis Calculus provides effects such as anti-inflammation, anti-spasm, anti-hypoxia, anti-depression, sedation, lowering blood pressure, reducing fever, and promoting normalization of gallbladder functions.

Bear bile powder is the dried product derived from the bile of black bears in the family Ursidae, after undergoing cholecystostomy, is effective in clearing heat, detoxifying, soothing liver, improving vision, insecticidal properties and hemostatic effects, is considered a rare and precious traditional Chinese medicinal material, is found in many ancient prescriptions, and is mostly used clinically for treating liver, gallbladder and ophthalmology diseases. The main components of bear bile powder are bile acids and non-bile acids, the bile acids including conjugated bile acids, such as tauroursodeoxycholic acid (TUDCA) and taurochenodeoxycholic acid (TCDCA), unconjugated bile acids, such as ursodeoxycholic acid (UDCA) and chenodeoxycholic acid (CDCA), as well as small amounts of bile acids and deoxycholic acid, the non-bile acids including cholesterol, amino acids, bilirubin, proteins, and peptides, as well as trace amounts of metallic elements. Modern pharmacological studies have shown that bear bile powder provides pharmacological effects such as hepatoprotection, choleretic activity, antipyretic and analgesia, anti-virus and anti-tumor.

Heart failure is categorized in traditional Chinese medicine as such as “dyspnea”, “heart constriction”, “heart water retention”, and “heart failure”. The etiology and pathogenesis of the condition are intricate and complex, with the disease fluctuating rapidly, making it a challenging and stubborn disorder. From the perspective of traditional Chinese medicine, it is believed that the fundamental pathological mechanism of this disease involves a combination of deficiency and excess, where deficiency mainly involves the heart, spleen, and kidneys, while excess refers to water retention and blood stasis. In particular, symptons of yang deficiency in the heart and kidneys, water retention and blood stasis are present throughout the disease. In the treatment, reinforcing healthy qi to eliminate pathogenic factors is the main focus, in which reinforcing healthy qi focuses on benefiting qi and warming yang, and eliminating pathogenic factors focuses on draining water retention and transforming blood stasis.

The traditional Chinese medicine composition in the present disclosure enhances the efficacy of the medicine and broadens the therapeutic scope by ingeniously combining a variety of traditional Chinese medicine components simultaneously, which achieves concurrent regulation of qi, blood, yin and yang, and simultaneous treatment of internal organs, and provides effective therapeutic effects on heart failure. Specifically, each traditional Chinese medicine composition used in the present disclosure contains a variety of active components, which are complementary to each other, and are synergized under specific ratios to exert better efficacy to play an effective role in preventing or treating heart failure, which is demonstrated as follows:
(1) Enhancement of myocardial contractility and reduction of cardiac load, increase in cardiac output, improvement of blood pumping function, prevention and treatment of heart failure, and improvement of cardiac function;
(2) Improvement of blood rheology to reduce cardiac load, increase of oxygen supply to the myocardium, improvement of cardiac function, and reduction of ventricular hypertrophy;
(3) Regulation of immune function, improvement of immunity in patients with heart failure, improvement of patients' left ventricular diastolic and systolic function, delay of ventricular remodeling, improvement of microcirculation, and anti-ischemia of the myocardium; and
(4) Inhibition of production of endothelin and tumor necrosis factor, dilation of blood vessels, deceleration of heart rate, reduction of myocardial oxygen consumption, improvement of myocardial blood supply, and protection of cardiac function.

Preferably, in terms of parts by weight, the traditional Chinese medicine composition includes the following components: 51.6 parts of Semen Myristicae, 44.8 parts of Radix Vladimiriae, 103.6 parts of Fructus Chebulae, 53.2 parts of Stigma Croci, 22 parts of Bovis Calculus, and 11.6 parts of bear bile powder.

Preferably, the heart failure includes chronic heart failure.

Preferably, a preparation method of the traditional Chinese medicine composition includes the following steps: mixing all components with a solvent, heating and refluxing 2 to 3 times with a reflux time of 1 to 3 hours, filtering, combining filtrates, and concentrating the filtrates.

In accordance with another aspect of the present disclosure, provided is a traditional Chinese medicine composition for treating heart failure, in which the traditional Chinese medicine composition includes following components in terms of parts by weight: 46-53.6 parts of Semen Myristicae, 42.4-49.2 parts of Radix Vladimiriae, 98.4-111.6 parts of Fructus Chebulae, 50.8-65.2 parts of Stigma Croci, 17.2-26 parts of Bovis Calculus, and 9.2-13.6 parts of bear bile powder.

Preferably, the traditional Chinese medicine composition includes following components in terms of parts by weight: 51.6 parts of Semen Myristicae, 44.8 parts of Radix Vladimiriae, 103.6 parts of Fructus Chebulae, 53.2 parts of Stigma Croci, 22 parts of Bovis Calculus, and 11.6 parts of bear bile powder.

### BRIEF DESCRIPTION OF THE DRAWINGS

FIG. 1 shows the results of the cardiomyocyte with heart failure characteristics (hereinafter referred to as model cardiomyocyte) activity test in Example 2 of the present disclosure.

FIG. 2 shows the test results of relative ejection fraction of mice in Example 3 of the present disclosure.

FIG. 3 shows the test results of the left ventricular thickness of the mice in Example 3 of the present disclosure.

### DETAILED DESCRIPTION OF THE EMBODIMENTS

For a better understanding of the solutions of the present disclosure by those skilled in the art, the technical solutions in the examples of the present disclosure are clearly and completely described and discussed below. Obviously, the examples described herein are only some of the examples of the present disclosure but not all of them.

### Example 1 Preparation of the Traditional Chinese Medicine Composition

The traditional Chinese medicine composition is prepared according to the following steps:

Mixing all components with solvent, heating and refluxing 2 to 3 times for 1 to 3 hours, filtering, combining filtrates, and concentrating the filtrates. The traditional Chinese medicine composition is obtained.

In the process mentioned above, the components (in terms of parts by weight) include 51.6 parts of Semen Myristicae, 44.8 parts of Radix Vladimiriae, 103.6 parts of Fructus Chebulae, 53.2 parts of Stigma Croci, 22 parts of Bovis Calculus, and 11.6 parts of bear bile powder.

### Example 2 Cellular Function Test of the Traditional Chinese Medicine Composition

Model cardiomyocytes induced to day 15 were selected and cultured for at least one month in T75 culture flasks or model cardiomyocytes were revived in T75 culture flasks and cultured for at least one month.

Model cardiomyocyte plating: the flasks were washed twice with 1640 culture medium, 8 mL of a digestive solution was added, it was digested at 37° C. until the cells were detached from a bottom of a T75 flask, stop digest solution 1640 basic culture medium was added, the cell suspension was collected into a 50 mL centrifuge tube, and was centrifuged at 210 g for 5 min. The supernatant was discarded after centrifugation, the cells were resuspended with revival solution, and 10 μL was taken for cell counting. Cells were plated in a 96-well plate at 20,000 cells per well with a liquid volume of 90 μL per well, leaving the last three wells without cells and adding an equal amount of revival solution in these wells.

After the third day of culture, the revival solution was discarded, the flasks were washed twice with 1640 culture medium, and the culture medium containing the medicine was added, which was set at three concentrations of high (10 μM), medium (3 μM), and low (1 μM). Equal volumes of cardiomyocyte culture medium were used as a negative control, and the dexamethasone injection (10 μM) was used as a positive control. Compound 028 indicates the traditional Chinese medicine composition in Example 1, and 049 and 056 indicate digoxin and spironolactone, respectively, which are commercially available frontline drugs.

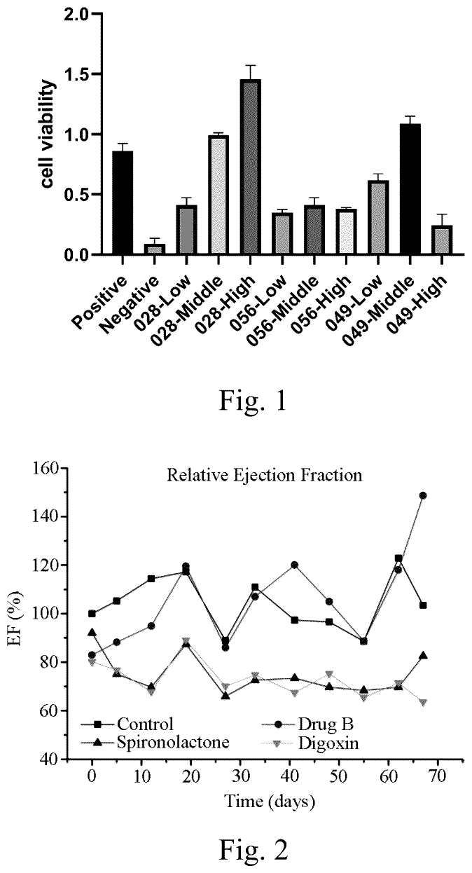

Model cardiomyocyte viability assay: four days after cell culture, viability assay reagents were added. Before using the PrestoBlue reagent, it was allowed to equilibrate to room temperature. Under light-protected conditions, Prestoblue reagent was added to the 96-well plate, followed by light-protected incubation at 37° C. for 1 h. The activity of the model cardiomyocytes was subsequently measured using a microplate reader, and the specific test results are shown in FIG. 1. Before using the microplate reader to measure, it needs to be turned on for 0.5 h in advance, allowing the machine to warm up, and the instrument was ensured to work correctly before measuring by checking the selected parameters of the test project, setting the wavelength (excitation and emission wavelengths of 560 mm and 590 mm, respectively), output format, and the default saving path, and confirming that all parameters are correctly set to ensure the normal function of the instrument.

According to FIG. 1, it is evident that the model cardiomyocytes treated with the traditional Chinese medicine extracts of the present disclosure exhibit better cellular activities than the model cardiomyocytes treated with the drugs digoxin and spironolactone at all three concentrations of high (10 μM), medium (3 μM), and low (1 μM), respectively. It indicates that the traditional Chinese medicine composition provided by the present disclosure is effective in increasing cardiomyocyte activity, thereby promoting myocardial oxygen supply, improving microcirculation and anti-ischemia in the myocardium, which may effectively treat heart failure and improve cardiac function.

### Example 3 Animal Experiments in Mice by Performing Ligation of Transverse Aortic Constriction (TAC)

Male KM mice were weighed and anesthetized by intraperitoneal injection of sodium pentobarbital (40 mg/kg). After anesthesia, the mice were fixed in a supine position on a mouse board, with the clavicle as the upper boundary, the xiphoid as the lower boundary, the left midaxillary line as the left boundary, and the anterior midline as the right boundary area for skin preparation. The mouth was secured with fine thread to maintain the airway. The mouse board on which the mice were fixed was placed horizontally on the table, with the neck illuminated by a cold light source i.e., a gooseneck lamp. A tongue spatula was used to gently pull the tongue out of the mouth, and then the spatula was held against the base of the tongue and lifted upward gently, revealing a clearly visible circular bright spot in the pharynx under the cold light, which indicated the opening of the trachea. A homemade endotracheal tube was inserted into the trachea and the ventilator was connect, with the respiratory rate being set at 90-100 breaths/min and a tidal volume of 0.3-0.4 ml. After successful tracheal intubation, the mice were repositioned to the right lateral recumbent position, and the left forelimb was fixed towards the head to fully expose the thorax.

The thorax was disinfected with iodine, and a layered thoracotomy was performed at the second intercostal space on the left side of the mice, with an incision size of about 7 mm. A spreader was used to expand the intercostal space, micro-forceps were utilized to separate the thymus and expose the aortic arch. A short segment of 6.0 silk suture was placed below the aortic arch between the brachiocephalic artery and the left carotid artery, a square knot was tied around the aortic arch for future use. A 27 G blunt needle tip was inserted into the knot and placed parallel to the artery, the knot was tightened to secure the needle and artery, another knot was tied, the needle was promptly removed, the excess suture ended next to the knot was cut off, completing the aortic arch constriction procedure. Animals in the sham-operated group underwent thoracotomy only without the constriction procedure, with the remaining steps identical to those of the model group.

Suturing needles with 4-0 sutures were used, layer by layer, to close the chest and sew the skin. The incision was disinfected after suturing. After suturing, a trial disconnection from the ventilator was conducted while retaining the tracheal tube. If the mice breathed stably and normally after disconnection, it allows complete removal from the ventilator; if the breathing of the mice is slow, the removal from the ventilator is prolonged appropriately.

To prevent hypothermia after surgery, the mice were placed on an electric heating pad, allowed to recover, and transferred to a rearing cage with drinking water. The number of animals that died in both experimental and postoperative groups was recorded, and autopsies were performed to determine the cause of death.

In the present experiment, the value of blood flow velocity greater than 2400 mm/s at the site of the aortic arch constriction was used as a selection criterion for the model, and the animals in the model group that met the selection criterion were included for further study, while the animals in the model group that did not meet the criterion were excluded from further study.

### Administering the Medicine

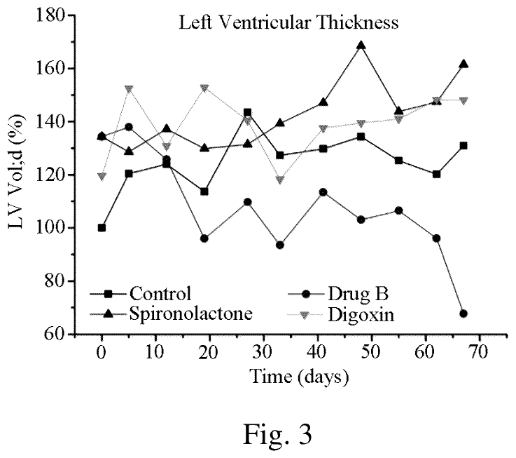

A total of 60 SPF-grade 4-week-old male Kunming (KM) mice were fed at a 12-hour light-dark cycle at an ambient temperature ranging from 20° C. to 25° C. The experimental animals were assayed weekly postoperatively, with no fewer than 10 mice per group at each time point. The mice in the sham-operated group were administered a blank solvent (control group), and the post-operative mice in the operated group were randomly divided equally into 3 groups, and were administered the traditional Chinese medicine combination of Example 1 (denoted as Drug B in FIGS. 2 and 3) as well as commercially available frontline medications for heart failure, i.e., digoxin and spironolactone, respectively. Based on clinical dose conversions, the concentration of Drug B was 25 mg/mL, the concentration of digoxin was 6.5 μg/mL, and the concentration of spironolactone was 100 μg/mL, with a daily gastric lavage volume of 200 μL.

### Observation and Testing

The survival time of mice was observed, and electrocardiogramography was performed on live mice.

Using an ultrasonic machine and the corresponding probe configured with a center frequency of 30 MHz and a standard frame rate of 449 fps, the experimental animals were sequentially subjected to hair removal from the chest area, isoflurane respiratory anesthesia and supine position fixation before testing. Left ventricular (hereinafter referred to as LV) M-mode echocardiography and two-dimensional images of the left ventricle were obtained in left parasternal short-axis section, and left cardiac long-axis section. In the long-axis and short-axis sections, the cardiac cycle is recorded at each measurement point, and the LV end-systolic internal diameter, LV end-diastolic internal diameter, LV posterior wall systolic thickness, LV posterior wall diastolic thickness, LV anterior wall end-diastolic thickness, LV anterior wall end-systolic thickness, the left ventricular/body mass index (LV/BMI), LV short-axis reduction rate, LV ejection fraction, LV diastolic volume, LV systolic volume, and left ventricular weight were obtained. The relative ejection fraction of mice was calculated based on the above data and is shown in FIG. 2. Also, the left ventricular thickness of the mice was measured, and the results of the left ventricular thickness test are shown in FIG. 3.

According to FIG. 2, it is evident that, compared to the mice treated with the drugs of spironolactone and digoxin, the mice treated with the traditional Chinese medicine composition of the present disclosure showed ejection fraction values closer to those of control group (the control group is a sham-operated group, in which the mice have normal cardiac function), which indicates that the traditional Chinese medicine composition of the present invention is effective in increasing the ejection fraction of the heart, and in improving the systolic and diastolic functions of the heart.

According to FIG. 3, it is evident that, compared to the mice treated with the drugs of spironolactone and digoxin, mice treated with the traditional Chinese medicine composition of the present disclosure showed a more significant reduction in left ventricular thickness, indicating that the traditional Chinese medicine composition of the present disclosure is more effective in treating heart failure and in improving of cardiac function.

The above examples are only used to illustrate the technical solution of the present disclosure rather than to limit the protection scope of the present disclosure. Although the present disclosure has been described in detail with reference to the above examples, a person of ordinary skill in the art should be aware that modifications or equivalent substitutions may be carried out to the technical solution of the present disclosure, these modifications or substitutions fall within the protection scope of the present disclosure.

1. A method of treating heart failure, comprising administrating to a subject an amount of a traditional Chinese medicine composition, wherein the traditional Chinese medicine composition comprises following components in terms of parts by weight:
46-53.6 parts of Semen Myristicae, 42.4-49.2 parts of Radix Vladimiriae, 98.4-111.6 parts of Fructus Chebulae, 50.8-65.2 parts of Stigma Croci, 17.2-26 parts of Bovis Calculus, and 9.2-13.6 parts of bear bile powder.

2. The method of treating heart failure according to claim 1, wherein the traditional Chinese medicine composition comprises following components in terms of parts by weight:
51.6 parts of Semen Myristicae, 44.8 parts of Radix Vladimiriae, 103.6 parts of Fructus Chebulae, 53.2 parts of Stigma Croci, 22 parts of Bovis Calculus, and 11.6 parts of bear bile powder.

3. The method of treating heart failure according to claim 1, wherein the heart failure comprises chronic heart failure.

4. The method of treating heart failure according to claim 1, wherein a preparation method of the traditional Chinese medicine composition comprises following steps: mixing all components with a solvent, heating and refluxing 2 to 3 times with a reflux time of 1 to 3 hours, filtering, combining filtrates, and concentrating the filtrates.

5. A traditional Chinese medicine composition for treating heart failure, wherein the traditional Chinese medicine composition comprises following components in terms of parts by weight:
46-53.6 parts of Semen Myristicae, 42.4-49.2 parts of Radix Vladimiriae, 98.4-111.6 parts of Fructus Chebulae, 50.8-65.2 parts of Stigma Croci, 17.2-26 parts of Bovis Calculus, and 9.2-13.6 parts of bear bile powder.

6. The traditional Chinese medicine composition for treating heart failure according to claim 5, wherein the traditional Chinese medicine composition comprises following components in terms of parts by weight:
51.6 parts of Semen Myristicae, 44.8 parts of Radix Vladimiriae, 103.6 parts of Fructus Chebulae, 53.2 parts of Stigma Croci, 22 parts of Bovis Calculus, and 11.6 parts of bear bile powder.
